(19) United States
(12) Patent Application Publication
(10) Pub. No.: US 2004/0158113 A1
(43) Pub. Date: Aug. 12, 2004

(54) Catalysts and process for converting fuel gases to gasoline

(76) Inventors: Girish Srinivas, Thornton, CO (US); Steven Charles Gebhard, Golden, CO (US); Theodore Sidney Hoover, Houston, TX (US)

Correspondence Address: Greenlee, Winner and Sullivan, P.C., Suite 201, 5370 Manhattan Circle, Boulder, CO, 80303, US

(21) Appl. No.: 10/360,536

(22) Filed: Feb. 6, 2003

(51) Int. Cl. C07C 2/58; B32B 5/02

(52) U.S. Cl. 585/721; 422/139; 422/224

(57) ABSTRACT

A process for alkylation of olefin-containing gases with low molecular weight branched alkanes to generate alkylate. The process involves the use of solid acid catalysts as alkylation catalysts. In a specific embodiment, the process employs a three-phase catalytic reactor where olefins present in the gas are reacted, preferably to extinction, with a large excess of liquid branched alkane.

### STATEMENT REGARDING GOVERNMENT-SPONSORED RESEARCH

This invention was funded at least in part by United States Government funding through the Department of Energy contract number DE-FG03-02ER83550. The United States government has certain rights in this invention.

### BACKGROUND OF THE INVENTION

Gasoline, diesel and other motor fuels are the single largest source of energy derived from petroleum in the United States. In 2000, Americans consumed 19.7 million bbl/day of petroleum of which 13.4 million bbl/day was used in the transportation sector (British Petroleum 2002). About 41% of each barrel of crude oil was converted into gasoline (British Petroleum 2002). In addition, the US produces less than 50% of the crude oil that is consumes.

Because of political and economic uncertainties in many of the countries that export oil to the United States, along with the large quantities of oil that need to be imported by the U.S., refining technologies that improve the yield and quality of gasoline obtained from each barrel of crude oil will increase U.S. energy independence by improving the efficiency of crude oil use.

The fluid catalytic cracking unit (FCCU) is central to gasoline manufacture in most refineries. The FCCU can use a variety of feed streams including gas oils from the atmospheric and vacuum columns or other heavy oils. The heavy oils contact hot catalysts and are cracked to more valuable lower molecular weight products. Overhead cracked vapors from the FCCU are first sent to a main fractionation section where heavies, cycle oils and naphtha are recovered. Overhead vapors from the fractionation section are sent to the vapor recovery unit (VRU). The VRU recovers the C₃/C₄ fraction (liquefied petroleum gas—LPG) and FCC gasoline (C₅⁺).

Overhead vapors from the VRU are referred to herein as the FCCU off-gas. Example off-gas compositions for two currently operating FCCUs are given in Table 1. This off-gas contains considerable amounts of hydrogen and light alkanes and alkenes and essentially all of the H₂S generated in the FCCU.

TABLE 1
Compositions of off-gas from two crackers (vol %).

| Component | | FCCU “#1” | FCCU “#2” |
|---|---|---|---|
| Off Gas Rate | SCF/bb1 | 289 | 180 |
| Hydrogen | H₂ | 29.24 | 7.88 |
| Nitrogen | N₂ | 9.82 | 8.73 |
| Carbon Monoxide | CO | 0.44 | 1.47 |
| Carbon Dioxide | CO₂ | 2.60 | 2.43 |
| Hydrogen Sulfide | H₂S | 0.63 | 0.06 |
| Methane | CH₄ | 28.27 | 37.69 |
| Ethane | C₂H₆ | 11.19 | 14.97 |
| Ethylene | C₂H₄ | 12.54 | 17.44 |
| Propane | C₃H₈ | 0.65 | 1.09 |
| Propylene | C₃H₆: | 2.76 | 3.91 |
| iso-Butane | i-C₄H₁₀: | 0.58 | 1.41 |
| n-Butane | n-C₄H₁₀ | 0.11 | 0.30 |
| iso-Butene | i-C₄H₈: | 0.29 | 0.57 |
| 1-Butene | 1-C₄H₈ | 0.21 | 0.48 |
| 1,3-Butadiene | C₄H₆ | 0.00 | 0.00 |
| trans-2-Butene | trans-C₄H₈: | 0.23 | 0.53 |
| cis-2-Butene | cis-C₄H₈ | 0.16 | 0.37 |
| iso-Pentane | i-C₅H₁₂ | 0.19 | 0.39 |
| n-Pentane | n-C₅H₁₂ | 0.00 | 0.00 |
| Hexane Plus | ≧C₆H₁₄ | 0.11 | 0.30 |
| Total | | 100.00 | 100.00 |

The alkenes (olefins) have chemical value but the off-gas is normally burned as refinery fuel gas because conventional recovery methods (such as cryogenics) are normally uneconomical.

There is a need in the art for an economical process for conversion of alkenes in the FCCU off-gas to chemically useful products. Of particular interest would be a process to generate alkylate from the FCCU off-gas.

Table 1 shows that in addition to ethylene and other light olefins, the FCCU off-gas contains considerable amounts of hydrogen. In addition the off-gas contains modest amounts of H₂S. The H₂S must be removed to provide economically useful product.

Total U.S. FCC capacity in 2002 was reported to be 5,646,161 bbl/day and typical inputs are about 90% of capacity (EIA 2003). Assuming that each FCC produces (on average) 228 standard cubic feet (SCF) of FCC-off gas that contains approximately 16% total olefins (mostly ethylene), approximately 200,000 bbl/day of alkylate could be produced if the olefins in the FCC off-gas were alkylated with isobutane. The potential increase in alkylate production by converting the FCC off-gas olefins would be approximately 200,000 bbl/day in the U.S. alone; worldwide, the potential could be nearly 500,000 bbl/day. With fuel gas having a value of $3/MM BTU, isobutane and n-butane costs of 36¢/gal, and alkylate being worth 55¢/gal, an estimated $5.6 MM/year value could be generated for a typical U.S. refinery by converting the FCCU off-gas olefins into alkylate.

In addition, the published MTBE manufacturing capacity in the U.S. is 240,800 bbl/day; thus, approximately 83% of the U.S. MTBE capacity could be replaced on a barrel for barrel basis with our new alkylation technology. Using the additional alkylate to replace MTBE could be a potential market because MTBE is falling out of favor for use in gasoline because of concerns with groundwater pollution from leaking underground storage tanks. While ethanol is currently being used as an MTBE replacement, ethanol has the disadvantage of increasing the Reid vapor pressure (RVP) of the gasoline, which increases fugitive hydrocarbon emissions. For example, when ethanol is blended into a gasoline with an RVP of 8.9 psi, the RVP of the ethanol-gasoline blend is increased to 9.5 psi (ethanol concentration of 10%). In addition, ethanol gasoline mixtures have a tendency to absorb water and cannot be stored for long periods of time and are therefore blended at the terminal.

Another factor affecting the alkylate market is that the allowable concentrations of aromatic hydrocarbons in gasoline are continually decreasing. This reduces the amount of reformate (one of the best sources of octane as shown by Table 2) that can be blended into the gasoline.

One of the reasons for decreasing the aromatics content of gasoline is that it reduces tail pipe emissions. For example, by reducing the aromatics content from 45% to 25%, CO and hydrocarbon emissions are both significantly decreased. A 25% limit on aromatics concentration

TABLE 2
Octane numbers for selected gasoline-blending
components.

| Component | RON | MON | RVP (psi) |
|---|---|---|---|
| LSR gasoline (C₅ to 180° F.)⁽²⁾ | 61.6 | 66.4 | 11.1 |
| LSR gasoline once through isom.⁽²⁾ | 81.1 | 83.0 | 13.5 |
| Hydrog. FCC gasoline (C₅ to 200° F.)⁽²⁾ | 81.7 | 91.2 | 14.1 |
| Hydrocracker prod C₆ to 190° F.⁽²⁾ | 73.7 | 75.5 | 3.9 |
| Alkylate from ethylene⁽¹⁾ | 89.0 | 97.0 | 9.8 |
| Alkylate from propylene⁽²⁾ | 87.3 | 90.8 | 5.7 |
| Alkylate from butylenes⁽²⁾ | 95.9 | 97.3 | 4.6 |
| Alkylate from mixed C₃⁼ and C₄⁼⁽²⁾ | 93.0 | 94.5 | 5.0 |
| Alkylate from pentenes (amylenes)⁽²⁾ | 88.8 | 89.7 | 1.0 |
| Reformate (94 RON)⁽²⁾ | 94.0 | 84.4 | 2.8 |
| Reformate (98 RON)⁽²⁾ | 98.0 | 86.5 | 2.2 |
| Reformate (100 RON)⁽²⁾ | 100 | 88.2 | 3.2 |
| Ethanol⁽³⁾ | 130 | 96 | 2.3 |
| Methyl tert-butyl ether (MTBE)⁽³⁾ | 118 | 100 | 8.0 |
| Ethyl tert-butyl ether (ETBE)⁽³⁾ | 118 | 102 | 4.1 |
| Methyl tert-amyl ether (TAME)⁽³⁾ | 111 | 98 | 2.6 |

⁽¹⁾RON/MON for 2,2-dimethylbutane, RVP = Pᵥₐₚ 100° F.
⁽²⁾Gary and Handwerk 1994
⁽³⁾RON/MON from Hochhauser 1994; RVP values are Pᵥₐₚ at 100° F.

is currently in the California Phase 2 gasoline requirements (Table 3). When the concentration of MTBE, other oxygenates or aromatics are reduced, the best way to make up octane is to blend in additional alkylate.

Alkylate blending stocks are composed of branched, paraffinic hydrocarbons and contain no aromatic hydrocarbons. Branched paraffins have high blending octane numbers (Table 2) and relatively low Reid Vapor Pressures (RVP) compared with other gasoline blending components such as light straight run (LSR) gasoline or isomerate. The low RVP of alkylate means that the octane number of gasoline can be increased without large increases in fugitive hydrocarbon emissions. In addition, it is easier to control the RVP of the fuel for summer and winter driving by blending in n-butane (RVP≈50 psi) if the gasoline has a low RVP.

TABLE 3
California Ph-2 Gasoline
Requirements (Hochhauser 1994)

| Property | Limit/liter |
|---|---|
| RVP (psi) | 6.96 |
| Sulfur (ppm) | 40 |
| Aromatics (vol %) | 25 |
| Olefins (vol %) | 6 |
| T₉₀ (° C.) | 149 |
| T₅₀ (° C.) | 99 |
| Oxygen (wt %) | 1.8-2.2 |
| Benzene (vol %) | 1.0 |

Current commercial alkylation processes are based on the reaction between isobutane (from the FCCU, n-butane isomerization or outside purchase) and C₃-C₅ olefins streams (largely butenes) using concentrated H₂SO₄ or HF as catalysts (Meyers 1997). The H₂SO₄ process requires that the refinery be relatively close to an acid supply because large volumes of spent and fresh acid are handled (Hammershaimb, et al. 1992). The lowest practical reaction temperature for H₂SO4 alkylation is about −5° C. because there are very large power requirements for mixing the viscous acid with the hydrocarbon mixture (Gary and Handwerk 1994). HF alkylation requires less power because mixing is more easily accomplished (sometimes by gravity alone) and HF regeneration is part of the unit. Typical process conditions for H₂SO₄ alkylation and HF alkylation are given in Table 4. Because of safety concerns with concentrated acids (especially HF) there has been a significant amount of research to develop a solid acid catalyst that would be a suitable substitute for the liquid acids.

TABLE 4
HF and H₂SO₄ alkylation conditions.

| | HF | H₂SO₄ |
|---|---|---|
| Isobutane (vol % rxn zone) | 30-80 | 40-80 |
| External i-C₄/olefin | 3-12 | 3-12 |
| Internal i-C₄/olefin | | 50-1,000 |
| Total contact time (min) | 8-20 | 20-30 |
| Reactor temp (° C.) | 16-46 | 2-16 |
| Reactor acid conc (wt %) | 80-95 | 88-95 |
| Acid in emulsion (vol %) | 25-80 | 40-60 |

Solid acid catalyzed alkylation R&D has been ongoing for over 10 years, and while processes and catalysts for alkylation have been reported, there was no installed capacity as of year 2000. The difficulty appears to be largely associated with rapid deactivation of the catalysts by fouling and coking. Hydrogen in the feed improves resistance to deactivation and is used in UOP's Alkylene process for the alkylation of conventional feedstocks (Kojiman and Kocal 1995; Hydrocarbon Processing, 2000). There remains a significant need in the art for economically viable processes for alkylation of low molecular weight olefins in gas streams.

### SUMMARY OF THE INVENTION

The present invention provides a process for alkylation of olefin-containing gases with low molecular weight branched alkanes to generate alkylate. The process involves the use of solid acid catalysts having H₀ (Hammet function) less than −12 as alkylation catalysts. In a specific embodiment, the process employs a three-phase catalytic reactor where olefins present in the gas are reacted, preferably to extinction, with a large excess of liquid branched alkane.

In a specific embodiment, the invention relates to a catalytic process where the C₂-C₄ olefins, particularly those contained in fluid catalytic cracking unit (FCCU) off-gas are converted into valuable alkylate useful, for example, for blending into gasoline.

In this alkylation process, FCCU off-gas is passed into a catalytic reactor where the olefins are reacted preferably to extinction with a large excess of branched alkane (e.g., isobutane or iospentane) in the presence of a solid acid catalyst. The catalytic reaction is preferably a three-phase reactor, such as a bubble column reactor. A trickle-bed reactor or a three-phase fluidized bed reactor can also be employed. The non-olefinic gases (H₂, normal alkanes, etc.) in the off-gas pass through the reactor and rejoin the general refinery fuel gas stream. The process is preferably operated such that it is compatible with the process pressure of the FCCU off-gas. The temperature of the reaction is between −20° C. and +150° C., and preferably between −10° C. and +30° C. In this method the pressure can be adjusted to keep the branched alkane and alkylate in the liquid phase.

After reaction, alkylate product and excess branched alkane are separated from the catalyst and alkylate product is separated from excess branched alkane, non-olefinic gases and any remaining olefin gases (which may be recycled through the reactor to obtain desired level of reaction) Gases and excess branched alkane can be removed from alkylate by known methods, for example, fractionation methods can be applied to recover the alkylate. Excess branched alkane is preferably recycled to the reactor (recycled branched alkane can for example be mixed with incoming branched alkane).

Preferred catalysts comprise modified mixed metal oxides that have acid sites available for alkylation. Precious metals such as Pt and Pd or other hydrogenation catalysts can be included to increase catalyst lifetime. Prolonged catalyst life may be achieved by hydrogenation of the fouling precursors in the presence of H₂ before they build up to damaging levels. The process of this invention employs strong solid acids to effectively alkylate isobutane with ethylene. Strong solid acids are those having H₀ of −12 or less. Hydrogen sufficient for hydrogenation of fouling precursors may be contained in the off-gas or may in whole or in part be added to the rector.

This invention, in a specific embodiment, provides a solid acid alkylation process to convert low molecular weight olefins (primarily ethylene) primarily into C₆-C₈ alkylate that is suitable for blending into gasoline. In this embodiment the preferred low molecular weight branched alkane used in the alkylation process is isobutane. In another specific embodiment, the invention provides a solid acid alkylation process to alkylate low molecular weight olefins (primarily ethylene) with iso-pentane.

The invention is further illustrated by the Drawings, Detailed Description and the Examples.

### BRIEF DESCRIPTION OF THE DRAWINGS

FIG. 1A and B schematically illustrate an FCCU-interfaced alkylation process of this invention. FIG. 1A illustrates an example FCCU system to generate FCCU off-gas. FIG. 1B illustrates a system for catalytic alkylation. The FCCU off-gas from the system of FIG. 1A optionally passes through a sulfur-removal system, e.g., an amine unit, prior to introduction into the three-phase catalytic reaction system of FIG. 1B. The system of FIG. 1B provides a fractionation system downstream of the alkylation reactor to return olefin-free fuel gas to the refinery, recycle the unreacted isobutane back to the alkylation reactor, and recover the alkylate product.

FIG. 2 is a graph showing strength measured as −H₀ of selected solid and liquid acids. The more negative the value of the Hammet function (H₀), the stronger the acid.

FIG. 3 is a process flow diagram for an experimental batch alkylation reactor used in Examples herein.

FIG. 4 is a gas chromatogram of products generated during ethylene alkylation with isobutane using sulfated zirconia catalyst (˜10 hr contact time). See Example 1.

FIG. 5 is a graph of the yield various alkylation products and total yield for ethylene alkylation using sulfated zirconia catalysts as in Example 1.

FIG. 6 is a gas chromatogram of the products generated during propylene alkylation with isobutane using sulfated zirconia catalyst as in Example 2.

### DETAILED DESCRIPTION OF THE INVENTION

The method of this invention is exemplified in a process for generating alkylate from the off-gas from an FCCU by alkylation with branched alkane, particularly isobutane. The olefin-containing FCCU off-gas, if necessary, is first treated to remove hydrogen sulfide (H₂S), for example, by passage through an amine-unit. The process is run under pressure conditions where the low molecular weight alkane is a liquid, for example, if isobutane is used, the pressure may be greater than 50 psig. After reaction, alkylate and excess (unreacted) branched alkane are separated from the catalyst, e.g., in a settler/filter. Alkylate product is then recovered and excess branched alkane is recycled back to the alkylation reactor.

The process herein uses a solid acid catalyst and is particularly beneficial for use with olefin containing gas streams that may also contain hydrogen, such as the off-gas of FCCU. Once hydrogen sulfide has been removed from the olefin-containing gas, a hydrogenation co-catalyst, such as Pt or Pd, can be employed to increase the lifetime of the solid acid catalyst. Hydrogen present in the feed catalytically removes fouling precursors, in situ, from the surface of the catalyst.

The process of this invention is further particularly useful for olefin-containing gas streams where the total olefin concentration in the gas is low, less than about 50% by volume, (more preferably less than about 25% by volume olefins). The process and catalysts of his invention are particularly useful for generating alkylate from olefin containing gas streams where ethylene is the predominant olefin, (e.g., 75% by volume or more of the olefins present).

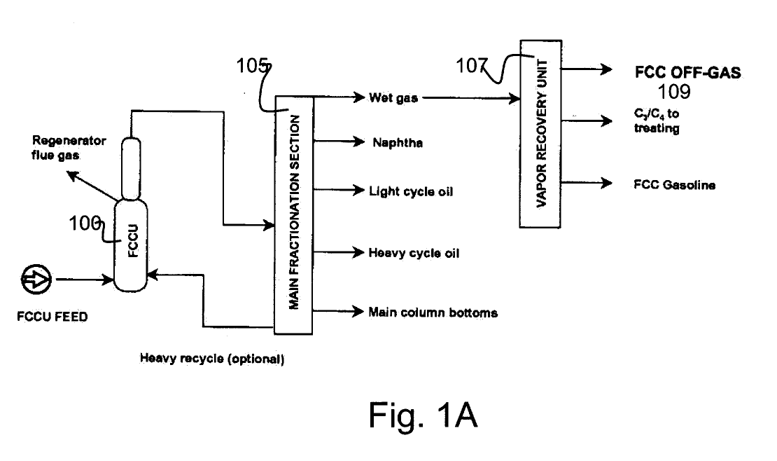

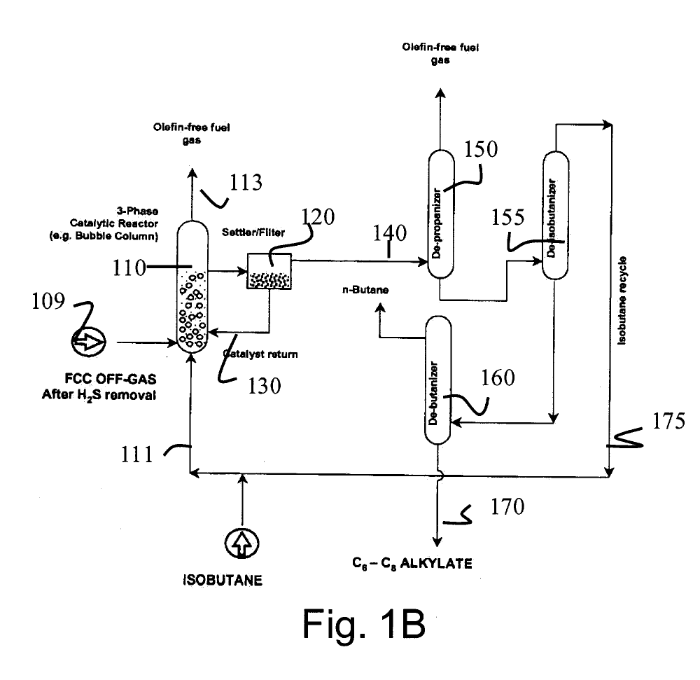

In a preferred embodiment, process FCCU off-gas is, if necessary, first treated in an amine unit to remove H₂S. The amine-treated gas is then passed into a three-phase catalytic reactor where the olefins present in the gas are reacted to extinction with a large excess of liquid branched alkane, e.g., isobutane. The process is run under conditions where the branched alkane, e.g., isobutane is a liquid (P>50 psig). The non-olefin gases (H₂, normal alkanes, etc.) pass through the reactor and pass into the general refinery fuel gas stream or are used elsewhere in the refinery. A hydrogenation catalyst such as Pt and Pd can be used as a co-catalyst to increase catalyst lifetime. The process is operated at pressures between 50-500 psig, such that it is compatible with the pressure of the FCCU off-gas feed. The pressure can be adjusted to keep the isobutane and alkylate in the liquid phase. The temperature of the reaction is between −20° C. and +150° C., preferably between −10° C. and +30° C. After reaction, alkylate product and excess isobutane are separated from the catalyst in a settler/filter, the catalyst is returned to the reactor, and the liquid is sent to fractionation to recover alkylate product. A schematic of the process integrated with the FCCU is shown in FIGS. 1A and 1B.

FIGS. 1A and 1B together show the inventive process interfaced with an FCCU, such as would be employed in a refinery. FIG. 1A illustrates an exemplary FCCU as is known in the art which comprises the FCCU (100) with a fractionation unit (105) and a vapor recovery unit (107). FCC off-gas (109) exits the vapor recovery unit. Any art-known FCCU that generates FCC off-gas can be employed in this invention. FIG. 1B schematically shows the new process elements that are incorporated downstream of the FCCU for carrying out the off-gas alkylation of this invention.

After reaction, alkylate product and excess isobutane are separated from the catalyst in a settler/filter (120), the catalyst is returned (130) to the reactor, and the liquid (140) is sent to fractionation (150, 155, 160) to recover the alkylate (170). Isobutane is recycled (175). Alternatively, the catalytic reactor used for the alkylation process may be a trickle-bed reactor or a 3-phase fluidized bed reactor. Those of ordinary skill in the art can adapt known three-phase reactors for use in the alkylation process of this invention.

The preferred branched alkane for reacting with olefins for producing gasoline-blending stocks is isobutane. Isobutane gives branched products that have high octane numbers. Also, because isobutane does not contain a β-carbon, the extent of undesirable isomerization is reduced. Isopentane is also a useful isoalkane for this process.

FCCU off-gas contains ethylene, propylene and a small amount of butylenes and therefore the primary alkylation reactions are:

C₂H₄+iso-C₄H₁₀→C₆H₁₄ “C₆ alkylate”

C₃H₆+iso-C₄H₁₀→C₇H₁₆ “C₇ alkylate”

C₄H₈+iso-C₄H₁₀→C₈H₁₈ “C₈ alkylate”

The reactions that it is desirable to minimize are hydrogenation of the olefin (which reduces alkylate yield), olefin polymerization (which can foul the catalyst), and cracking of the alkylate product because this makes lower molecular weight products. All of these reactions, including the desired alkylation reactions, are catalyzed by acids, thus the detailed nature of the catalyst and the operating conditions can control the relative rates of these competing reactions. Control of the composition of the alkylate product can be done by controlling catalyst composition, reaction temperature and pressure, residence time and the concentrations of the reactants (including H₂).

Isoalkanes (isoparaffins) and aromatics can be alkylated with olefins using Bronsted acid catalysts. Examples include the industrially important alkylation of butylenes and amylenes with isobutane using liquid H₂SO₄ and HF catalysts as well as other catalysts that have been mostly used in laboratory studies such as HC1-AlCl₃, HF-SbF₅, and HF-BF₃, and other highly acidic systems. Some of these mixtures (e.g. HF-SbF₅) are superacids and have Hammet acidities (H₀) less than −12 which is the strength of 100% H₂SO₄. Such liquid superacid systems are corrosive, reactive, and react dangerously with water and therefore are not extensively used on an industrial scale. As a result the vast majority of both academic and industrial research and development has been focused on the development of solid acid catalysts.

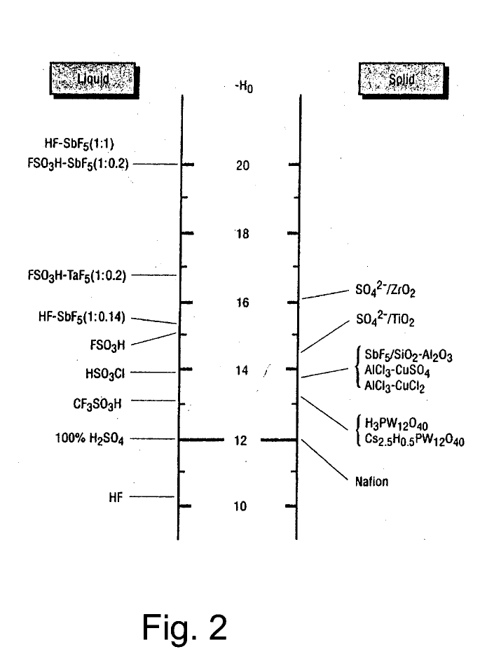

FIG. 2 compares the acidity of several liquid and solid acid systems. The more negative the value of the Hammet function H₀ the stronger is the acid. FIG. 2 shows −H₀, so the higher up the graph, the stronger the acid. For example, concentrated H₂SO₄ has an H₀ of about −12, whereas HF-SbF₅ has an H₀ of −20. This means that the HF-SbF₅ catalyst is roughly 100 million times more acidic than 100% H₂SO₄ (i.e. the equilibrium constant for HA→H⁺+A⁻ is 10⁸ times larger). These extremely acidic protons can protonate molecules that are difficult or essentially impossible to protonate with acids of conventional strength. Several of the solid acids have Ho values between about −13 (the polyoxometalates) and −16 (sulfated zirconia) which are considerably stronger than 100% H₂SO₄. This is important because neither H₂SO₄ nor HF is strong enough for ethylene alkylation with isobutane (Kennedy 1958). Thus, acids with H₀<−12 are needed for the alkylation of FCCU off-gas because the most abundant olefin is ethylene. FIG. 2 shows that there are a number of solid acid catalysts that can be used for ethylene alkylation.

The acidity and catalytic activity of solids have been extensively studied (for reviews see Corma 1995; Misono and Okuhara 1993) and not surprisingly, many attempts have been made to develop solid acid catalysts that could perform isobutane/butene alkylation as well as H₂SO₄ or HF, but without the hazards associated with the liquid acids. The primary problems so far have been rapid catalyst deactivation apparently due to fouling by polyolefinic materials on the surface. These are formed because olefin polymerization is also acid catalyzed (Olah and Molnar 1995). Compounding the problem is the fact that both strong and weak acid sites will catalyze polymerization, but only the strongly acidic sites can catalyze alkylation, thus selectivity for alkylate can be lost well before the catalyst becomes completely inactive.

Yang 1974 and 1975 report a process for alkylation of olefins (designed for gas streams containing high concentrations of C₃-C₅ olefins) in which a solid-acid catalyst is employed. The catalyst contains a small amount of precious metal (e.g., Pt) as a hydrogenation catalyst. The system reported has two separate reactors, one for alkylation and one for regeneration of the catalyst. Alkylation occurs in a first reactor at temperatures below 100° F. A small slipstream of catalyst/hydrocarbon slurry is constantly withdrawn from the first reactor and sent to a regenerator reactor, which operates at 200-250° F. Hydrogen gas is added to the regenerator to remove polyolefinic deposits on the catalyst before they completely foul the catalyst. Regenerated catalyst is then returned to the alkylation reactor (Zhang et al. 1997). The process can be operated continuously.

Another approach that has been used is to add a third component that acts as a solvent to remove the polyolefinic deposits as they form. An example of this is the use of liquids and liquefied gases operated at near supercritical conditions (Ginosar et al. 1998). In this application, conventional feedstocks were used (mostly butenes), temperatures were as high as 150° C. and pressures were between 600 and 1200 psi.

In the process of this invention both alkylation and hydrogenation reaction can take place in a single reactor such that a separate catalyst regeneration reactor is not required.

Table 5 shows data for the alkylation of isobutane with ethylene using different catalysts. While all of the catalysts in Table 5 are difficult to handle and hazardous, the results shown indicate that if an acid that is stronger than HF or H₂SO₄ is available, then ethylene alkylation is possible. None of the catalysts in Table 5 are practical for commercial application because of the catalyst handling problems.

TABLE 5
Alkylation of isobutane with ethylene (Kennedy 1958).

| Catalyst | Temp (° C.) | Remarks |
|---|---|---|
| H₂SO₄ (100%) | | No catalysis (H₀ < −12) |
| HF (anhydrous) | | No catalysis |
| Alkyl fluoride + | −80 and | No hexanes except 2,3 |
| BF₃ | higher | dimethylbutane (DMB) |
| AlCl₃ + HCl | −15 to 0 | No isomerization of 2,3-DMB |
| AlCl₃ + HCl or AlCl₃ + | 25 | Isomerization of 2,3-DMB |
| Ethyl chloride | | |
| BF₃ + HF + H₂O | 40-45 | Extensive isomerization of |
| (trace) | | 2,3-DMB |
| Ni + HF + BF₃ | 20-30 | Isomerization plus some |
| | | disproportionation |

Both HF and H₂SO₄ acids are too weak to effectively alkylate isobutane with ethylene and the liquid acid systems in Table 5 are impractical. On the other hand, there are many solid acids just as strong as the liquid acid systems listed in Table 5. Thus, the catalysts of this invention are those modified and unmodified mixed metal oxides, zeolites, aluminas, silica-aluminas and other inorganic solids that exhibit acidic behavior, a few examples of which are shown in Table 6.

TABLE 6
Catalysts.

Alkylation Function
Zeolite-β (acidic)
Zeolite-Y (acidic)
Cs_{2.5}H_{0.5}PW₁₂O₄₀
H₃PW₁₂O₄₀
Cs_{2.5}H_{0.5}PW₁₂O₄₀/Carbon
H₃PW₁₂O₄₀/Carbon
SO₄²⁻/ZrO₂
SO₄²⁻/TiO₂
SO₄²⁻/Nb₂O₅

Solids such as H₃PW₁₂O₄₀ and Cs_{2.5}H_{0.5}PW₁₂O₄₀, supported antimony pentafluoride (SbF₅/SiO₂-Al₂O₃), and sulfated titania and sulfated zirconia (H₀≈−16) are 1,000-10,000 times stronger than 100% H₂SO₄. The catalysts for our alkylation process comprise metal oxides and mixed metal oxide catalysts, both modified and unmodified, as well as other inorganic solid acids including but are not limited to, those listed in Table 6. All of these catalysts are known to exhibit strong acidity and several have been shown to be active for alkylation of isobutane with butenes (e.g., zeolite-β, CS_{2.5}H_{0.5}PW₁₂O₄₀, H₃PW₁₂O₄₀, SO₄²⁻/ZrO₂) (Molnaret al. 2001; Ohgoshi et al. 1996; Okuhara et al. 1996; Santana and Akgerman 2001; Song and Sayari 1996; Sun et al 2000; Del Rossi et al 1995; Essayem et al. 2001).

Table 7 shows a list of co-catalysts that can be incorporated into the solid acid catalyst to add a hydrogenation capability to the alkylation catalyst. All of these catalysts have hydrogenation activity, but there are two main classes, metals and sulfur resistant catalysts. The first class includes the traditional precious metal hydrogenation catalysts, for example, Pt and Pd, other “platinum-group metals,” and certain transition metals such as nickel and cobalt. They are very active for hydrogenation, but are rapidly and irreversibly poisoned by H₂S. Another type of hydrogenation catalyst is the class consisting of metal oxides based on molybdenum and tungsten that have been sulfided by pretreatment with H₂S including: Co/MoO₃, Ni/MoO₃, Co/WO₃ and Ni/WO₃. These are catalysts used in hydrodesulfurization (HDS), which have moderate hydrogenation activity, are sulfur tolerant, and are used when only small concentrations of H₂S are present in the FCCU off gas.

TABLE 7
Hydrogenation-catalysts

Pt, Pd, Ru, etc. (for H₂S free gas)
Ni, Co (metallic form for H₂S free gas)
Sulfided-Co/MoO₃ Ni/MoO₃, Co/WO₃ and
Ni/WO₃ (sulfur tolerant catalysts)

Approximately 50% of the gasoline produced by the refinery comes from the FCCU. Hydrotreating, reforming, and thermal processing of the atmospheric and vacuum distillates contribute most of the rest. The fraction of the crude that is not utilized in making gasoline, middle distillates (kerosene, diesel, and jet), fuel oils and lube feedstocks, consists of gases and high molecular weight residuum. Considerable research and development is ongoing to increase the yield of motor fuels from residuum and this will be vital to increasing the fraction of each barrel of crude oil that is used for fuel production. This invention is concerned with increasing crude utilization with a process that uses the valuable olefins from the FCCU off-gas stream in the refinery that would otherwise be used as fuel gas.

Conversion of the FCCU off-gas olefins into alkylate by the inventive process has the potential to produce approximately 200,000 bbl/day of alkylate in the United States (based on a total U.S. catalytic cracking capacity of 5,646,161 bbl/day (EIA 2003) and a gas generation rate of 228 SCF/bbl of FCCU feed) Up to 500,000 bbl/day could be produced worldwide. This will increase the utilization of each barrel of crude for gasoline production. For example, the total crude oil consumption in the U.S. in 2000 was 19.7 MM bbl/day, and the net gasoline production (produced minus exported) was about 8.1 MM bbl/day (British Petroleum 2002). Thus, approximately 41% of each crude barrel is converted into gasoline that is used in the U.S. Thus, by adding 200,000 bbl/day of alkylate from the inventive process to the gasoline pool, the amount of gasoline is increased to 8.3 MM bbl/day. The conversion is now increased to 42% of each barrel of crude producing gasoline. This represents a significant increase in crude oil utilization. If MTBE use is phased out in the future, additional alkylate capacity will become even more important. For example, the published MTBE manufacturing capacity in the U.S. is 240,800 bpd; thus 83% of the MTBE currently produced could be replaced on a barrel-per-barrel basis using our FCCU off-gas alkylate.

As MTBE plants are phased out, the isobutenes produced by the FCCU must be processed in either the alkylation or a modified MTBE unit to produce gasoline (isobutenes are currently used to make MTBE). These isobutenes could augment the off-gas feed alkylation process to produce additional alkylate. In some cases, the capital expenditure could be an incremental number over a base case revamp of the existing HF or H₂SO₄ alkylate processes.

Converting the olefins in FCCU off-gas into alkylate adds value to a stream that would otherwise be used as fuel gas. The alternative of recovering the olefins by refrigeration is normally uneconomical. For example, there are several refineries that recover the ethylene and other light olefins in a cryogenic plant, and one of these plants is considering shutting the cryogenic gas plant down because it cannot compete economically with ethylene production from LPG or naphtha cracking.

Preliminary economic calculations for adding the alkylation process of this invention to an FCCU have been performed. The upgrade was determined with a basis of 40,000 barrels of FCCU feed. Fuel gas (assuming that the heating value taken out by alkylation of the olefins must be replaced) was priced at $3/MM Btu. Isobutane and normal butane were priced at 36¢/gal, and alkylate was priced the same as premium gasoline @ 55¢/gal (normally alkylate is valued somewhat higher than premium gasoline as determined by each refinery's LP). These calculations indicate that the additional revenue from an upgrade incorporating the process of this invention would be $5.6 MM/year.

### EXAMPLES

### Experimental Methods

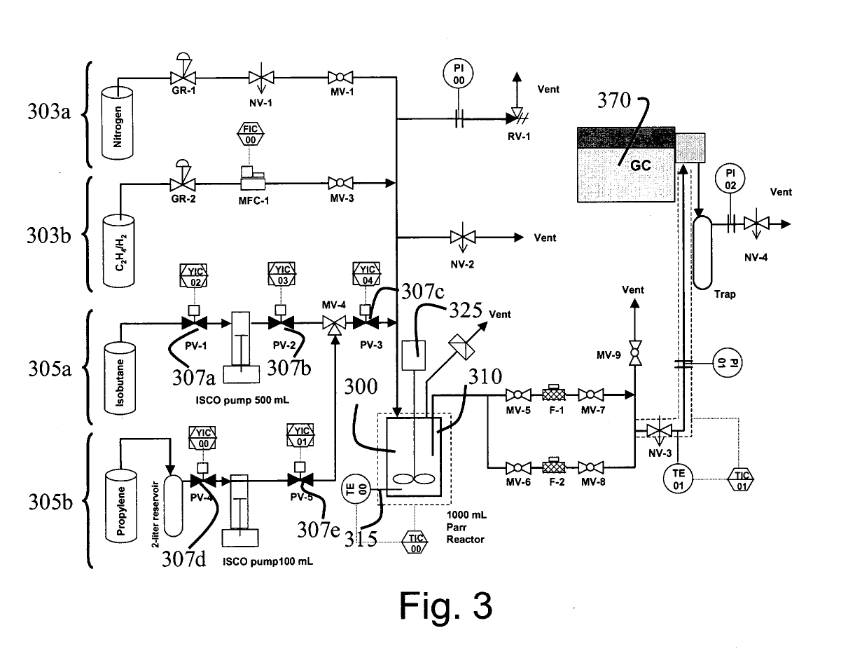

Alkylation experiments were performed in a batch mode using a one-liter, Parr brand, mechanically stirred, autoclave with the solid acid catalyst (in powder form) suspended in liquid isobutane. An overall process and instrumentation diagram is given in FIG. 3. The autoclave reactor (300) is equipped with gas (303a-b)and liquid inlets (305a-b) with appropriate pumping as needed, a dip tube outlet (310, for sampling the reactor contents), a thermowell (315), and a mechanically sealed stirrer (325). There is a separate port for a rupture disk (1000 psi) and the inlet gas manifold is equipped with a pressure gauge, a pressure transducer, and a manual relief valve that is set at 500 psig. The Parr reactor has a volume of 1000 mL and is made of stainless steel.

Coiled copper tubing is wrapped around the outside of the reactor body to cool the Parr autoclave. Cold, automotive-type antifreeze solution is circulated through the copper tubing using a chiller to maintain the reactor at a chosen reaction temperature. The temperature of the contents of the reactor is measured using a thermocouple located in the thermowell. A stainless steel dip tube is connected to heat-traced tubing that is used to transfer liquid from the autoclave to the gas chromatograph for analysis. The heat-traced sample transfer line (335) is maintained at a temperature of about 150° C.

For experiments with propylene, both the isobutane and propylene are transferred to the reactor as liquefied gases under pressure using ISCO syringe pumps. There are two pumps: a 1000 mL capacity pump that is used for isobutane, and a 500 mL pump used for propylene (or 1 -butene in some experiments). The ISCO pumps are filled from commercial cylinders of isobutane, propylene or 1-butene that are equipped with dip tubes (these hydrocarbons are shipped in liquid form).

The experimental apparatus is also equipped with electronic mass flow controllers to meter in ethylene gas, hydrogen gas and nitrogen gas. Nitrogen is used for general purging and pressurization operations. Hydrogen is added as a reactant for some experiments to maintain catalyst activity. Ethylene is metered into the reactor as a gas after the reactor has been charged with catalyst and liquid isobutane. A considerable amount of ethylene can be added because it is readily soluble in liquid isobutane. Typical experimental pressures are between 150-500 psig and typical experimental temperatures are between −20° C. and 100° C.

In a typical experiment, calcined catalyst is placed in the empty reactor. The reactor lid is then clamped in place and the system is purged with nitrogen gas at ambient pressure and temperature. After approximately 10 min of purging at a flow rate of about 1 liter/min, a small amount of hydrogen flow is started (ca. 25 mL/min) so that the gas in the reactor contains 4% or less H₂. The reactor is then pressurized to 100 psig above the planned experimental pressure and the system is leak checked with a hand-held flammable gas detector. The small amount of hydrogen in the system makes even very small leaks easy to detect.

Once the system has been determined to be leak free, the pressure is reduced and the system is again flushed with nitrogen to remove traces of the hydrogen used for the leak test. The reactor is then pressurized to 150 psig with nitrogen to prevent the isobutane or propylene from flash evaporating when they are transferred into the reactor using the ISCO pumps. A pressure of 150 psig is somewhat higher than the vapor pressure of propylene at room temperature and is well above the vapor pressures of isobutane or 1-butene. Once the reactor has been filled with a known volume of isobutane and the contents have cooled to the desired experimental temperature (e.g. 0° C.), the olefin (ethylene, propylene or butylene) is added.

Approximately 400 mL of liquid isobutane is used in the experiments and is dispensed using a 1000 mL ISCO syringe pump. This permits dispensing a precisely known quantity of liquid isobutane into the Parr autoclave reactor. Once the isobutane has been transferred, the reactor is again allowed to cool to the desired reaction temperature. When the isobutane is cold, 50-100 mL of propylene (butylene for some experiments) is dispensed into the reactor using the smaller ISCO pump. The exact amount used depends on the specific experiment, but the amount is accurately known. These tasks are done using the pneumatic valves (PV-1 through PV-5, 307a-e) shown in FIG. 3; each line is isolated/opened as required. In the experiments where ethylene is used, a measured mass of gas is added to the reactor. The mass of ethylene is known because the molar flow rate of ethylene can be calculated from the volumetric flow rate that is controlled by the mass flow controller. The mass of ethylene added is then equal to the molar flow rate multiplied by the flow time multiplied by the molecular weight of ethylene. In the experiments where hydrogen is added, it is added before the ethylene. Finally, nitrogen gas is added to the reactor to bring the contents of the reactor up to the desired experimental pressure. Once all of the reactants are in the reactor, the mechanical stirrer is started and contents of the reactor are periodically sampled.

The reaction between the olefins and isobutane will produce branched C₆-C₈ hydrocarbons with some higher molecular weight alkylate hydrocarbons (C₉ and up). All of the alkylation products are much less volatile than isobutane or the feedstock olefins (ethylene or propylene) and remain dissolved in the liquid isobutane (especially under elevated pressure conditions). In order to measure the quantities of the hydrocarbons formed by alkylation, it is necessary to sample the liquid phase during the course of the reaction. Because the system is under pressure and the isobutane and propylene would flash vaporize if the pressure were abruptly reduced, the reactor liquid cannot be simply drained and sampled with a gas chromatographic syringe. Therefore we designed a sampling system that is attached to a dip tube that extends down into the liquid phase inside the reactor. The contents of the reactor can then be sampled while the system remains pressurized.

In our sampling setup, a liquid sample is obtained by temporarily shutting off the stirrer (this minimizes the amount of catalyst that can be drawn up into the dip tube) and then a small stream of liquid is taken through a filter to a needle valve where it is flash vaporized into a heated (ca. 150° C.) ⅛ inch stainless steel line that goes to the gas chromatograph (370, FIG. 3). The dip tube in the reactor is made from 1/16 inch stainless steel tubing and has a small sintered filter element attached to the end, which is completely immersed in the liquid isobutane. Because all of the liquid is vaporized, all of the isobutane, unreacted olefin and alkylate products present in the liquid are analyzed by the GC in the vapor phase. The GC analysis is done in an a conventional manner using a heated gas sampling valve that injects a 1 mL sample of vapor directly into the GC column (silica gel packed column). The GC uses a flame ionization detector (FID). The liquid flow rate out of the sampling system is very small and therefore obtaining 10 or more samples does not appreciably affect the concentration of any of the components in the reactor. For example, a vapor flow rate of 25 mL/min corresponds to a liquid sample flow rate of about 0.1 mL/min. For example, 20 samples that require flowing liquid out of the reactor for 5 min each, corresponds to the removal of 10 mL of liquid (approximately 2% of the volume of the contents of the reactor). After the GC injection has been made, the sample line is closed and the stirrer is turned back on. The pressure falls slightly during sampling (ca 5 psi drop) and is compensated for by adding back nitrogen.

After the experiment, the pressure is slowly relieved to allow the isobutane and unreacted olefin to slowly vaporize. This is done with the reactor chilled to approximately 0° C. (or lower) to minimize evaporation of any alkylate product. After the pressure has been relieved, the reactor is opened and the liquid alkylate product is collected and filtered to remove catalyst particles. The clear alkylate is then analyzed by gas chromatography/mass spectrometry (GC/MS).

### Example 1

### Alkylation of Ethylene with Isobutane

Sixty grams of sulfated zirconia catalyst (Saint-Gobain Norpro, Akron, OH) were used in an ethylene alkylation experiment. The catalyst was ground and sieved to a size between 1.1 mm and 0.08 mm. Prior to being loaded into the reactor, the catalyst particles were calcined for 10 hours in air at 550° C. in a muffle furnace. After calcining, the catalyst was allowed to cool in the muffle furnace and then stored in a desiccator prior to use. The catalyst was then placed in the reactor, the reactor purged with nitrogen, leak tested and then charged with 400 mL of liquid isobutane (approx. 4 gram-moles) as described above. The 400 mL of isobutane and the reactor body were then cooled to 0° C. Ethylene was added to the system as a pressurized gas at a flow rate of 311 standard cubic centimeters per minute (sccm) for one hour. This corresponds to adding 0.83 gram-moles (gmole) of ethylene to the reactor. The catalyst plus liquid mixture was mechanically stirred with an electric stirrer, and the contents of the reactor were sampled every hour for 14 hours using the on-line gas chromatograph described above.

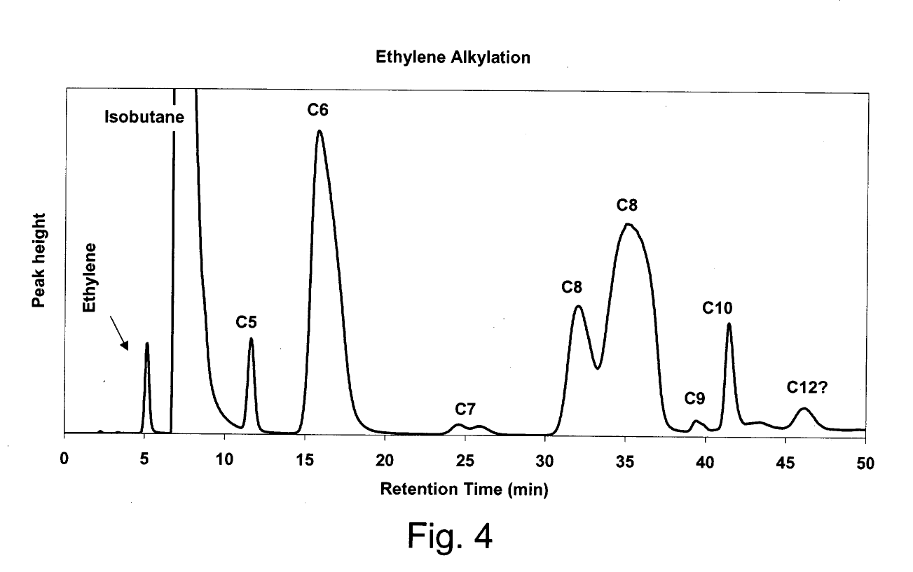

FIG. 4 is a typical gas chromatogram showing the composition of the contents of the reactor during ethylene alkylation with isobutane. The catalyst was a commercially available sulfated zirconia. The reaction temperature was 0° C. and the pressure was 200 psig. The predominant products are C6 alkylate (dimethylbutanes) and C8 products (isooctanes). Both alkylation and oligomerization/polymerization reactions are acid-catalyzed (Olah and Molnar 1995), and because the carbon chain grows via oligomerization/polymerization, some of the hydrocarbons formed during alkylation have more carbon atoms in them than the simple sum of the reactants. For example, ethylene (C2) alkylation with isobutane (C4) produces mostly dimethylbutanes (C6) but also gives octane isomers (C8) and smaller amounts of even higher hydrocarbons. This type of chemistry is observed in all acid catalyzed alkylation reactions including the commercial HF and H₂SO₄ processes where butylenes (C4) and isobutane (C4) produce branched C9, C10, etc. hydrocarbons in addition to the isooctane products (C8). The predominant products were of ethylene alkylation with isobutane using the sulfated zirconia catalyst were C6 and C8 alkylate (branched paraffins).

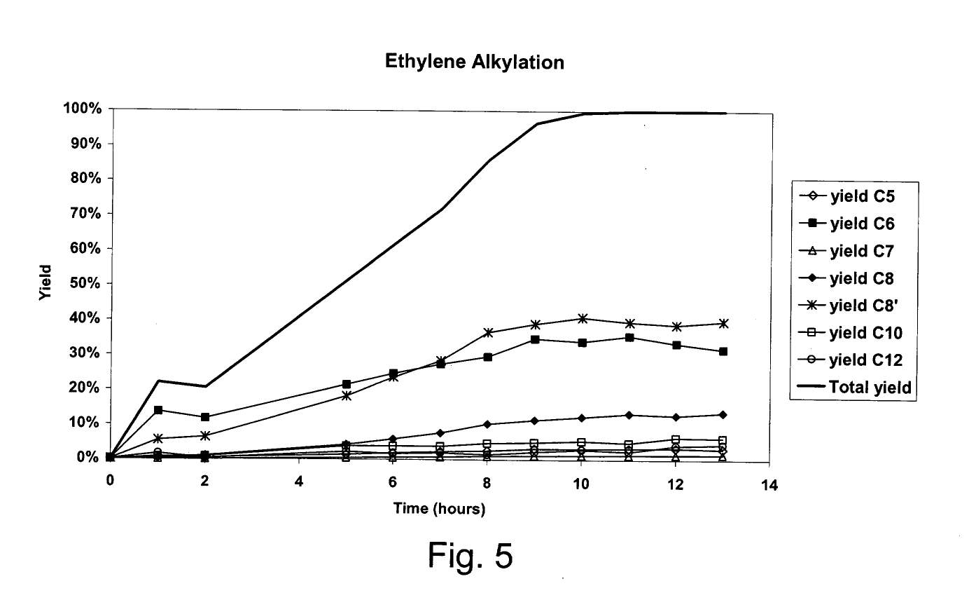

FIG. 5 shows ethylene conversion vs. time and indicates that 100% of the ethylene had reacted (total conversion=100%) by 10 hours. The graph in FIG. 5 also shows the product distribution and changes in the product distribution during this time. The fact that chain growth accompanies alkylation enhances the inventive process because it permits the production of octane isomers in addition to C6 isomers. By 10 hours, ethylene alkylation with isobutane had produced an alkylate product that contained approximately 34% (by moles) C6 and 41% C8 branched hydrocarbons. The results also indicate that by controlling the contact time with the catalyst, the composition of the products can be controlled—moderately longer contact times appear to favor modest chain growth increasing the yield of C8 alkylate.

Table 8 shows a summary of the gas chromatography/mass spectrometry (GC/MS) analysis of the alkylate product from the alkylation of ethylene with isobutane using a sulfated zirconia catalyst at a temperature of 0° C. The alkylate product contains some dissolved isobutane starting material (16.55%). This is not unusual because butanes are added to gasoline to adjust its vapor pressure and consequently, the solubility of butanes is substantial in C6-C9 hydrocarbons. The concentration of isobutane would be reduced (if desired) in the deisobutanizer column in the actual process (see FIG. 1B). The right-most column of Table 8 and of Table 9 shows the alkylate compositions renormalized with isobutane excluded. Most of the alkylate is C8 isoparaffins (57.64%) followed by C6 isoparaffins (30.88%). The detailed analysis indicates that most of the C8 isoparaffins are trimethylpentanes and dimethylhexanes. The C6 isoparaffins are primarily dimethylbutanes. The Research Octane Number (RON) calculated for the alkylate was 83.7 and the Motor Octane Number (MON) for the alkylate was calculated to be 82.4.

Table 9 shows the results for a similar experiment done where hydrogen gas was present in the alkylation reactor to give a hydrogen/ethylene molar ratio of approximately 1.7, which is approximately the average hydrogen/ethylene ratio for the two example FCCU gas streams listed in Table 1. Table 9 shows that the yield of C6 (dimethylbutanes) was increased from 30.88% to 44.49% while the yield of C8 (dimethylhexanes and trimethylpentanes) was decreased slightly from 57.64% to 48.07%. Importantly, adding hydrogen appears to have reduced the amount of higher molecular weight (C10 and C11) isoparaffins from 5.57+0.72=6.29% without hydrogen to 3.19% with hydrogen. These changes increased the calculated RON to 94.7 and the calculated MON to 91.7.

Significantly, in neither case were there significant concentrations if straight chain alkanes (paraffins) and no olefins, napthenes (cycloalkanes) nor aromatic hydrocarbons were produced; all of the produce was branched alkanes (isoparaffins).

TABLE 8
Summary of GC/MS analysis of alkylate produced in Example 1.

| | | | | | | Isoparaffin |
|---|---|---|---|---|---|---|
| | Paraffin | Isoparaffin | Olefin | Napthene | Aromatic | w/o isobutane |
| | (LV %) | (LV %) | (LV %) | (LV %) | (LV %) | (LV %) |
| C4 | 0.06 | 16.55 | 0.00 | | | |
| C5 | 0.00 | 0.72 | 0.00 | 0.00 | | 0.88 |
| C6 | 0.00 | 25.25 | 0.00 | 0.00 | 0.00 | 30.88 |
| C7 | 0.00 | 0.76 | 0.00 | 0.00 | 0.00 | 0.93 |
| C8 | 0.00 | 47.13 | 0.00 | 0.00 | 0.00 | 57.64 |
| C9 | 0.00 | 0.59 | 0.00 | 0.00 | 0.00 | 0.72 |
| C10 | 0.00 | 4.55 | 0.00 | 0.00 | 0.00 | 5.57 |
| C11 | 0.00 | 2.76 | | 0.00 | | 3.38 |
| Total LV % | 0.06 | 98.31 | 0.00 | 0.00 | 0.00 | |
| unidentified | 1.63 | | | | | |
| RON | | 83.70 | | | | |
| MON | | 82.40 | | | | |

TABLE 9
Summary of GC/MS analysis of alkylate
produced in Example 1 with added hydrogen.

| | | | | | | Isoparaffin |
|---|---|---|---|---|---|---|
| | Paraffin | Isoparaffin | Olefin | Napthene | Aromatic | w/o isobutane |
| | (LV %) | (LV %) | (LV %) | (LV %) | (LV %) | (LV %) |
| C4 | 0.16 | 44.07 | 0.00 | | | |
| C5 | 0.00 | 1.16 | 0.00 | 0.00 | | 2.08 |
| C6 | 0.00 | 24.81 | 0.00 | 0.00 | 0.00 | 44.49 |
| C7 | 0.00 | 0.52 | 0.00 | 0.00 | 0.00 | 0.93 |
| C8 | 0.00 | 26.81 | 0.00 | 0.00 | 0.00 | 48.07 |
| C9 | 0.00 | 0.00 | 0.00 | 0.00 | 0.00 | 0.00 |
| C10 | 0.00 | 1.78 | 0.00 | 0.00 | 0.00 | 3.19 |
| C11 | 0.00 | 0.69 | | 0.00 | 0.00 | 1.24 |
| Total LV % | 0.16 | 99.84 | 0.00 | 0.00 | 0.00 | |
| unidentified | 0.00 | | | | | |
| RON | | 94.70 | | | | |
| MON | | 91.70 | | | | |

### Example 2

### Alkylation of Propylene with Isobutane

The FCCU off gas also contains propylene (Table 1); so experiments with propylene alkylation were performed. Forty-three grams of the sulfated zirconia catalyst used in Example 1 were used in the propylene alkylation experiment. The catalyst was ground and sieved to a size between 1.1 mm and 0.08 mm. The catalyst particles were calcined for 6 hours in air at 550° C. After calcining, the catalyst was allowed to cool in the muffle furnace and then stored in a desiccator prior to use. The catalyst was then placed in the Parr reactor and the reactor was purged with nitrogen. After leak testing, a charge of 400 mL of liquid isobutane (approx. 4 gram-moles) was added. The 400 mL of isobutane and the reactor body were then cooled to 0° C., and 46 grams of propylene was dispensed into the reactor from a separate high-pressure syringe pump. The propylene was transfer as liquefied gas under pressure. This volume of liquid propylene corresponds to 0.6 gram-moles (gmole) added to the reactor. The catalyst plus liquid mixture was mechanically stirred with an electric stirrer, and the contents of the reactor were sampled at 1, 3 and 22 hours using the on-line gas chromatograph. The experimental temperature was 0° C. and the pressure was 200° C., the experimental temperature and pressure were the same as those used in the ethylene experiment of Example 1.

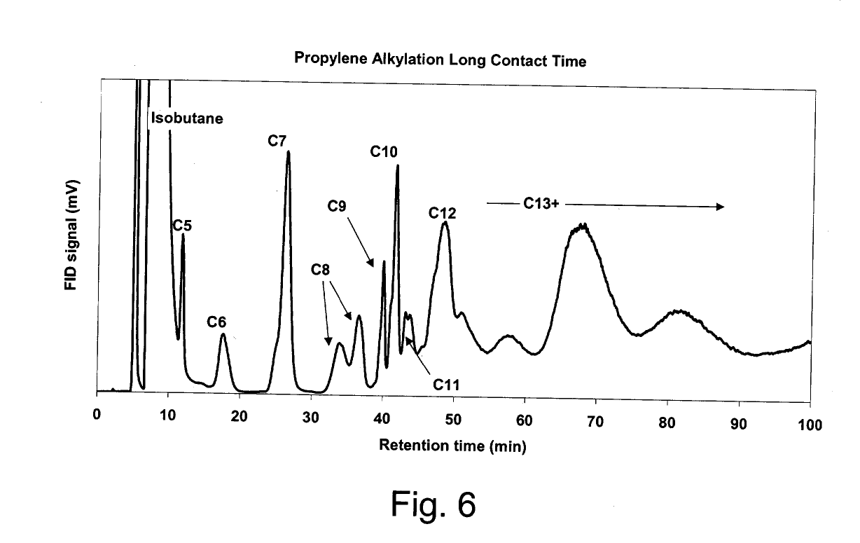

FIG. 6 shows a typical gas chromatogram for this experiment. The predominant product was C7 alkylate with approximately half of that amount was C8 alkylate. Lesser amounts of C9 and C10 alkylate were also formed. A considerable amount of higher molecular weigh alkylate (ca. C13) was produced when propylene was the olefin that was not observed when ethylene was the olefin used. In general (as expected) there was a greater percentage of higher molecular weight hydrocarbons formed when propylene was alkylated compared to ethylene. This result is consistent with the fact that propylene is somewhat more reactive than ethylene and that propylene has an additional carbon atom. As was the case with ethylene, some chain growth took place; however propylene (C3) alkylation with isobutane (C4) produces mostly C7 products.

Both ethylene and propylene are present in the FCCU off-gas (Table 1). Thus, alkylation of FCCU off-gas with isobutane will produce a mixture of branched paraffinic hydrocarbon products with molecular weights between about 72 (C5) and 170 (C12), with a peak in the molecular weight distribution around 114-142 (C8-C10). Other gas streams that contain low molecular weight olefins are also suitable feedstocks for alkylation with the inventive process. In these cases, the product distribution will depend on the concentrations of the olefins.

Those of ordinary skill in the art will appreciate that materials (catalysts and co-catalysts), methods (methods for preparation of catalyst, methods for gas analysis, fractionation methods, etc.) and apparatus (catalytic reactors) other than those specifically exemplified herein can be employed in the practice of this invention without resort to undue experimentation. In particular, any three-phase catalytic reactor can be employed or readily adapted for use with the catalysts of this invention for olefin alkylation. Branched alkanes other than isobutane can be employed to generate alkylate. Sources of olefin-containing gas other than FCCU off-gas can be treated by the methods of this invention using catalysts herein to generate alkylate. All art-known equivalents of the materials, methods and apparatus exemplified herein are intended to be encompassed by this invention. All references cited herein are incorporated by reference herein to the extent that they are not inconsistent with the disclosure herein. References cited herein provide details of art-known materials, methods and apparatus and, in particular, provide details of FCCU.

### References

British Petroleum (2002) U.S. Energy Statistics at www.bp.com.

Corma, A. (1995) “Inorganic Solid Acids and Their Use in Acid-Catalyzed Hydrocarbon Reactions,” Chem. Rev., 95, 558-614.

Del Rossi, K. J., Jablonski, G. A., Kresge, C. T., Juehl, G. H., Marler, D. O., Rav, G. S. and Rose, B. H. (1995) “Supported Heteropoly Acid Catalysts,” U.S. Pat. No. 5,475,178. Assigned to Mobil Oil Corp.

(EIA 2003) Energy Information Administration, U.S. Department of Energy at http://www.eia.doe.gov/oil gas/petroleum/info glance/refineryops.html

Essayem, N., Taarit, Y. B., Gayraud, P. Y., Sapaly, G. and Naccache, C. (2001) “Heteropolyacid Catalysts for Hydroisomerization of n-Hexane: Effects of Alkali Salt Modification,” J. Catal., 204, 157-162.

Gary, J. H. and Handwerk, G. E. (1994) Petroleum Refining, Technology and Economics, 3^{rd} ed. Dekker.

Ginosau, D. M.; Fox, Robert V.; Kong, and Peter C. (2000) “Solid Catalyzed Isoparaffin Alkylation at Supercritical Fluid and Near Supercritical Fluid Conditions,” U.S. Pat. No. 6,103,948.

Hammershiamb, H. U., Imai, T., Thompson, G. J, and Vora, B. V. “Alkylation,” (1992) Kirk-Othmer Encyclopedia of Chemical Technology, vol 2, John Wiley and Sons, New York.

Hochhauser, A. M. (1994) “Gasoline and Other Motor Fuels,” (1994) in Kirk-Othmer Encyclopedia of Chemical Technology, 4^{th} ed., vol 12, J. I. Kroschwitz and M. Howe-Grant (eds.) John Wiley and Sons, New York.

Hydrocarbon Processing (2000) “Special Report: Refining Processes 2000, Alkylaton, UOP Alkylene Process,” p. 89.

Kennedy, R. M. (1958) “Catalytic Alkylation of Paraffins with Olefins,” in Catalysis, Vol VI, P. H. Emmett (ed.) Reinhold Publishing Corp. New York.

Kojiman, M. and Kocal, J. A. (1995) “Regeneration of a Modified Alkylation Catalyst with Hydrogen,” U.S. Pat. No. 5,391,527.

Kresge, C. T., Marler, D. O., Rav, G. S., and Rose, B. H. (1994) “Supported Heteropoly Acid Catalysts,” U.S. Pat. No. 5,366,945.

Meyers, R. A. (1997) Handbook of Petroleum Refining Processes, 2^{nd} ed., McGraw Hill.

Misono, M. and Okuhara, T. (1993) “Solid Superacid Catalysts,” Chemtech, November, 23-29.

Molnar, A., Beregszaszi, T., Fudala, A., Nagy, J. B., Konya, Z. and Kiricsi, I. (2001) “The Acidity and Catalytic Activity of Supported Acidic Cesium Dodecatungstophosphates Studied by MAS, NMR, FTIR, and Catalytic Test Reactions, J. Catal, 202, 379-386.

Ohgoshi, S., Kanai, J., and Sugimoto, M. (1994) “Process for Alkylating an Isoparaffin with a Salt of a Heteropoly-Acid as a Catalyst,” U.S. Pat. No. 5,321,196.

Olah, G. and Molnar, A. (1995) Hydrocarbon Chemistry. Wiley Interscience.

Okuhara, T., Nishimura, T. and Misoni, M. (1996) “Novel Microporous Solid “Superacids”: Cs_{X}H_{3-X}PW₁₂O₄₀ (2≦×≦3),” Studies in Surface Science and Catalysis, vol. 101, 581-590, Elsevier Science B. V.

Santana, G.M. and Akgerman, A. (2001) “Alkylation of Isobutane with 1 -butene on a Solid Acid Catalyst in Supercritical Reaction Media,” Ind. Eng. Chem. Res. 40, 3879-3882.

Song, X. and Sayari, A. (1996) “Sulfated Zirconia-Based Strong Solid-Acid Catalysts: Recent Progress, Catal. Rev.—Sci. Eng., 38(2) 329-412.

Sun, W., Zhao, A., Guo, C., Ye, X. and Wu, Y. (2000) “Study of the Alkylation of Isobutane with n-butene over WO₃/ZrO₂ Strong Solid Acid. 1. Effect of the Preparation Method, WO₃ Loading and Calcination Temperature,” Ind. Eng. Chem. Res., 39, 3717-3725.

Yang, C. J. (1974) “Hydrocarbon Alkylation Process Using Catalyst Regeneration,” U.S. Pat. No. 3,851,004.

Yang, C. J. (1975) “Isoparaffin Alkylation Process with Periodic Catalyst Regeneration,” U.S. Pat. No. 3,893,942.

Zhang, S. Y-F., Gosling, C. D., Sechrist, P. A., and Funk, G. A. (1997) “Dual Regeneration Zone Solid Catalyst Alkylation Process,” U.S. Pat. No. 5,675,048.

We claim:

1. A catalytic process for alkylating olefins in an olefin containing gas, which comprises the step of:
(a) contacting an olefin-containing gas with one or more low molecular weight branched alkanes in the presence of a solid acid catalyst having a Hammett acidity (H₀) of less than −12 such that the olefins in the olefin-containing gas are alkylated by the one or more low molecular weight branched alkanes to generate an alkylate product.

2. The process of claim 1 further comprising the step of separating the alkylate product formed in step (a) from any remaining low molecular weight branched alkanes and any remaining olefin.

3. The method of claim 1 wherein the solid acid catalyst is selected from the group consisting of Zeoliteβ (acidic) Zeolite-Y (acidic), Zeolite ZSM-5 (acidic), Cs_{2.5}H_{0.5}PW₁₂O₄₀, H₃PW₁₂O₄₀, Cs_{2.5}H_{0.5}PW₁₂O₄₀/Carbon, H₃PW₁₂O₄₀/Carbon and other acidic polyoxometalates and heteropolyacids, sulfated ZrO₂, sulfated-titania and sulfated-niobium oxide, modified and unmodified sulfated metal oxides including sulfated-iron oxide, silica-aluminas, modified silica-aluminas, acidic aluminas, and other acidic inorganic oxides including natural materials such as acidic clays.

4. The method of claim 1 wherein the olefin containing gas predominantly contains ethylene.

5. The method of claim 1 wherein the branched alkane is isobutane.

5. The method of claim 1 wherein the branched alkane is isopentane.

6. The method of claim 1 wherein the olefin containing gas predominantly contains ethylene and the branched alkane is isobutane.

7. The method of claim 1 wherein an excess of low molecular weight branched alkane is employed.

8. The method of claim 1 further comprising the steps of separating unreacted branched alkane from the alkylate product and recycling the unreacted branched alkane for further reaction with olefin containing gas in the presence of the solid acid catalyst.

9. The method of claim 1 wherein the olefin containing gas also contains hydrogen gas.

10. The method of claim 9 wherein a hydrogenation catalyst is combined with the solid acid catalyst to promote the reaction of hydrogen with fouling precursors and minimize deactivation of the solid acid catalyst.

11. The method of claim 10 wherein the hydrogenation catalyst is a platinum group metal.

12. The method of claim 10 wherein the hydrogenation catalyst is Pd, Pt, or Ru.

13. The method of claim 10 wherein the hydrogenation catalyst is a transition metal

14. The method of claim 10 wherein the hydrogenation catalyst is Ni or Co.

15. The method of claim 10 wherein the hydrogenation catalyst is a hydrodesulfurization catalyst.

16. The method of claim 10 wherein the hydrogenation catalyst is cobalt promoted or nickel promoted molybdenum oxide or tungsten oxide.

17. The method of claim 10 wherein the hydrogenation catalyst is copper chromite.

18. The method of claim 1 wherein the olefin-containing gas is off-gas from a catalytic cracker used in petroleum refining.

19. The method of claim 10 wherein the solid acid catalyst is a sulfated metal oxide.

20. The method of claim 10 wherein the solid acid catalyst is sulfated zirconia.

21. The method of claim 10 wherein the low molecular weight branched alkane is isobutane.

22. The method of claim 10 wherein an excess of low molecular weight branched alkane is employed

23. An improved FCC unit which generates FCC off-gas containing one or more olefins and which comprises
(a) a catalytic reactor for receiving the FCC off-gas comprising a solid acid catalyst having a Hammett acidity (H₀) less than −12;
(b) an inlet for introducing a liquid phase low molecular weight branched alkane into the reactor;
(c) and a separator for separating alkylate product of the reaction of one or more olefins in the FCC off-gas with the low molecular weight branched alkane catalyzed by the solid acid catalyst from the reactor.

24. The FFC unit of claim 23 wherein the catalytic reactor is a three-phase reactor.

25. The FFC unit of claim 23 wherein the catalytic reactor is a bubble column reactor.

26. The FFC unit of claim 23 wherein the catalytic reactor is trickle-bed reactor.

27. The FFC unit of claim 26 wherein the catalytic reactor is mechanically agitated slurry reactor.

28. The FFC unit of claim 23 wherein the low molecular weight branched alkane is isobutane.

29. The FFC unit of claim 23 wherein the low molecular weight branched alkane is isopentane.

30. The method of claim 23 wherein an excess of low molecular weight branched alkane is employed

31. The FCC unit of claim 23 wherein the concentration of olefin in the FCC off-gas is 25% by volume of less.

32. The FCC unit of claim 23 wherein the wherein the solid acid catalyst is selected from the group consisting of Zeolite-β (acidic) Zeolite-Y (acidic), Zeolite ZSM-5 (acidic), CS_{2.5}H_{0.5}PW₁₂O₄₀, H₃PW₁₂O₄₀, Cs_{2.5}H_{0.5}PW₁₂O₄₀/Carbon and other acidic polyoxometalates and heteropolyacids, sulfated ZrO₂, sulfated-titania and sulfated-niobium oxide, modified and unmodified sulfated metal oxides including sulfated-iron oxide, silica-aluminas, modified silica-aluminas, acidic aluminas, and other acidic inorganic oxides including natural materials such as acidic clays.
